(19) United States
(12) Patent Application Publication
(10) Pub. No.: US 2024/0390178 A1
(43) Pub. Date: Nov. 28, 2024

(54) OCCLUSION ORTHOSIS

(71) Applicant: SA LU THE - STIFTUNG FUER SANFTE LUNGENTHERAPIEN, Boppard (DE)

(72) Inventor: Heike Nocker-Bayer, Boppard (DE)

(21) Appl. No.: 18/694,428

(22) PCT Filed: Sep. 22, 2022
(86) PCT No.: PCT/EP2022/076403

§ 371 Date: Mar. 22, 2024

(30) Foreign Application Priority Data
Sep. 23, 2021 (DE) 10 2021 004 806.7

(51) Int. Cl. A61F 5/56; A61C 5/00

(52) U.S. Cl. CPC A61F 5/566; A61C 5/007

(57) ABSTRACT

Monobloc orthosis splint (1) for preventing mouth-breathing in an individual while his temporomandibular joint is in the rest position, comprising a first U-form half splint for the upper jaw (2) and a second U-form half splint for the lower jaw (3): both the first and second U-form half splints being made, at least partially, of plastic material and comprising posterior parts (4, 5) intended to be positioned in the molar and premolar region (ISO 3950 teeth number 14-16:24-26:34-36:44-46) of the individual mouth, at least the posterior parts of the first and second U-form half splints being hermetically assembled, wherein the splint is devoid of proximal and distal protuberances.

### BACKGROUND OF THE INVENTION

The present invention relates to an orthosis, more particularly to an occlusion orthosis, for re-educating patients to nasal and deep abdominal breathing.

Without exception, new-born babies breath with their abdomens through the nose. In fact, young babies until the age of 3 or 4 months, have not yet developed the reflex to breathe through their mouths.

Mouth breathing, particularly during sleep, may develop in response to some type of blockage in the upper airway, like the nose or throat. This may be caused by something fairly harmless like for example a cold or an allergy reaction, or it could be caused by other more complex conditions.

Mouth breathing is common in individuals whose nasal passages are blocked or restricted. A deviated nasal septum or small nostril size can lead a person to breathe through their mouth instead of their nose. Furthermore, a problem with bite alignment can make it difficult to keep the mouth closed, both during the day and when sleeping.

Breathing through the mouth may become a habit which is very difficult to break, particularly in older children or adults who have been doing this over longer periods of time. Many things can lead to a mouth-breathing habit. A small child might get a cold and then simply continue breathing through his mouth when his nose clears.

It is generally estimated that up to 30-50% of modern adults breathe through the mouth, especially during the early morning hours.

Breathing through the mouth at all times, including when sleeping, can lead to several problems.

In the short term, mouth-breathing dries out of the mouth, removing the first defence against oral bacterial, leading to poor oral hygiene. In the long term, the negative effects of mouth-breathing are life-altering, particularly when the habit begins in childhood and goes unchecked. In fact, long uncorrected mouth-breathing is associated with worsening serious diseases like the Chronic Obstructive Pulmonary Disease (COPD), asthma, including allergic asthma, sleep apnoea, fatigue syndrome, allergies, hypertension, and long-lasting COVID-19 symptoms. Chronic mouth breathing may also be associated with greater risk for dental complications, such as decay and gum diseases, problems with jaw joints, speech and swallowing difficulties and enlarged tonsils and adenoids.

Furthermore, mouth-breathing is a typical characteristic of over breathing. When an individual over breathes, too much carbon dioxide is lost from the blood and this results in reduced oxygenation of tissues and organs.

US Patent Application Publication No. US 2020/0215384 A1 discloses an oral training appliance for myofunctional training. US Patent Application Publication No. 2015/0223968 A1 discloses a device for relieving airways obstruction disorders that comprises a protuberance to be positioned in the buccal cavity. US Patent Application Publication No. 2017/03121117 A1 discloses a device for sleep apnoea and snoring treatment. This prior art provides splints that affect the natural position or movement of diverse anatomical structures of the mouth. Further, these splints affect the appearance of their user's mouth. Therefore, they cannot be worn continuously on a daily basis, including during sleep.

There is therefore the need to provide an improved orthosis splint which can be worn continuously (up to twenty-four hours a day) under all circumstances, for re-educating an individual to breath primarily with the nose.

### SUMMARY OF THE INVENTION

The invention relates to a monobloc orthosis occlusion splint for preventing mouth-breathing in an individual while his temporomandibular joint is in the rest position, comprising a first U-form half splint for the upper jaw and a second U-form half splint for the lower jaw;
both the first and second U-form half splints being made, at least partially, of plastic material and comprising a posterior part intended to be positioned in the molar and premolar region (ISO 3950 teeth number 14-16; 24-26; 34-36; 44-46) of the individual mouth, at least the posterior parts of the first and second U-form half splints being hermetically assembled, characterized in that the splint is devoid of proximal and distal protuberances.

In another aspect, the invention relates to the use of the monobloc orthosis splint described above for urging breathing through the nose and/or for preventing breathing through the mouth in an individual.

In still another aspect, the invention relates to the use of the monobloc orthosis splint described above for reducing or stopping shortness of breath, gasping breath, shallow breathing, dyspnoea, and breathlessness; or for treating or reducing symptoms associated with Chronic Obstructive Pulmonary Disease (COPD), asthma, including allergic asthma, sleep apnoea, fatigue syndrome, allergies, hypertension, long-lasting COVID-19 symptoms and snoring.

In a further aspect, the invention relates to the monobloc orthosis splint (1), for use in reducing or stopping shortness of breath, gasping breath, shallow breathing, dyspnoea and breathlessness; or in treating or reducing symptoms associated with Chronic Obstructive Pulmonary Disease (COPD), asthma, including allergic asthma, sleep apnoea, fatigue syndrome, allergies, hypertension, long-lasting COVID-19 symptoms and snoring.

A further aspect of the present invention is a method for urging breathing through the nose in an individual or for preventing breathing through the mouth in an individual.

In still a further aspect, the invention relates to a method for reducing or stopping in an individual shortness of breath, gasping breath, shallow breathing, dyspnoea, and breathlessness; or for treating or reducing in an individual symptoms associated with Chronic Obstructive Pulmonary Disease (COPD), asthma, including allergic asthma, sleep apnoea, fatigue syndrome, allergies, hypertension, long-lasting COVID-19 symptoms and snoring, comprising the individual using the monobloc orthosis splint described above.

The monobloc orthosis splint of the invention enables the static and dynamic occlusion while the temporomandibular joint is in the rest position. Even under effort or while moving in the daily life or during sport activities, the monobloc splint of the invention prevents the lower jaw from moving downwards and, accordingly, from opening lips and/or mouth. The monobloc orthosis splint of the invention keeps the mouth closed, leaves the larynx free and it presses the tongue, in a light and pleasant way, against the palate. In this way, the airflow is directed through inhalation and exhalation via the nose, along the soft palate, the uvula and the tongue into the trachea.

The structure of the monobloc orthosis splint of the invention enables a comfortable use. The individual can wear it twenty-four hours a day while sleeping, performing daily life tasks or under effort during sport activities. The individual maintains full control and can anytime get the monobloc orthosis splint out of the positioning to open the mouth. It is in fact instrumental that the user maintains the feeling that he or she can open the mouth at any time during wearing of the monobloc orthosis splint.

The person skilled in the art would appreciate that, while inducing mouth closure and thus nasal breathing, the splint disclosed herein allows an essentially natural movement of the tongue inside the buccal cavity. Among others, that is attained by the splint essentially resembling the gum's contour and by it being devoid of proximal protuberances. Further, the lack of distal protuberances allows a natural closure of the lips, and an overall natural position of all the parts of the mouth. Allowing a natural position of the mouth is essential for patient's compliance and comfort, as the disclosed splint can be prescribed for continuous use, and in some cases it might be worn for years. In addition, not affecting the outward appearance of the mouth is also an essential aspect.

### DESCRIPTION OF THE DRAWINGS

FIG. 1 is a perspective front view at an angle from the bottom of an embodiment of the monobloc orthosis splint of the invention;

FIG. 2 is a perspective rear view at an angle from the top (upper jaw) of the same embodiment;

FIG. 3 is a perspective rear view at an angle from the bottom (lower jaw) of the same embodiment;

FIG. 4 is a lateral (left) perspective view of the same embodiment;

FIG. 5 is a lateral (right) perspective view of the same embodiment;

FIG. 6 is a lateral (right) perspective view photo of the same embodiment, identifying the positioning of the monobloc orthosis splint with respect to the teeth of the user (ISO 3950 teeth number 23-27 and 33-37).

### DETAILED DESCRIPTION OF THE INVENTION

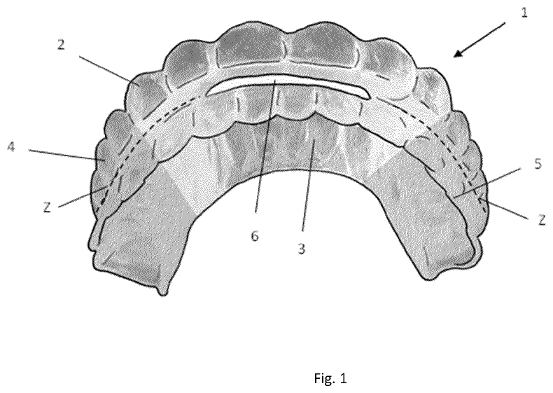

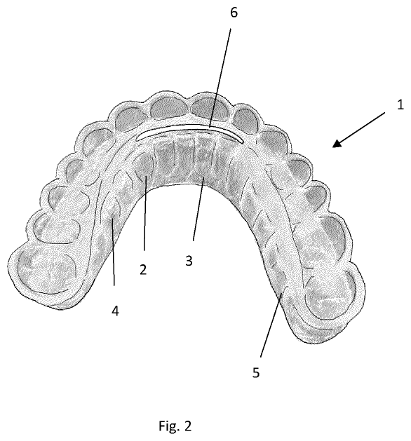

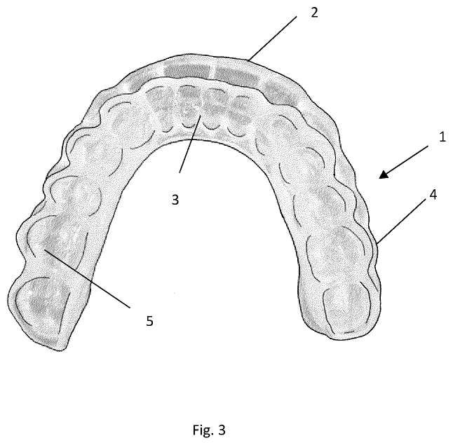

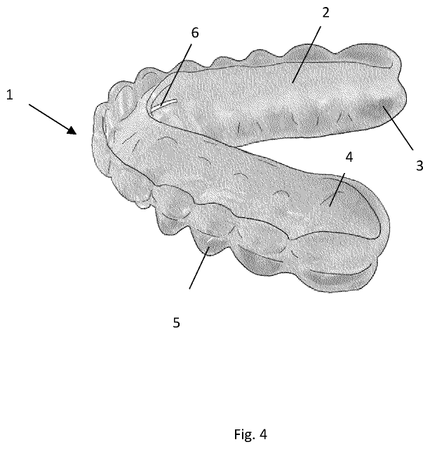

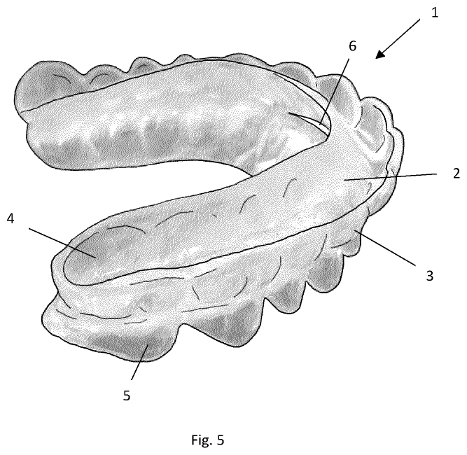

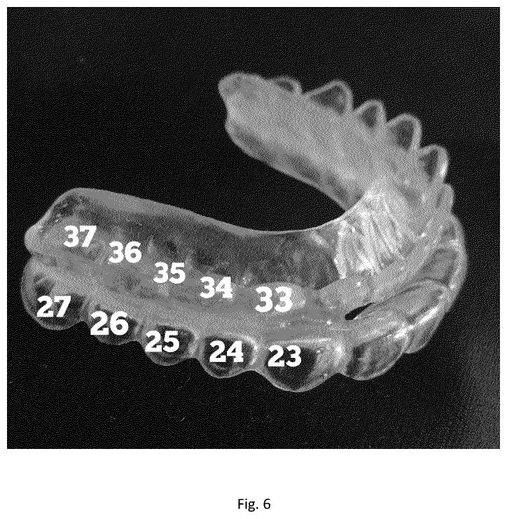

With reference to FIGS. 1 to 6, the monobloc orthosis splint (1) of the invention comprises a first U-form half splint (2) for the upper jaw and a second U-form half splint (3) for the lower jaw of an individual. Both the first and second U-form half splints (2,3) are made, at least partially, of plastic material and comprise a posterior part (4,5) intended to be positioned in the molar and premolar region (ISO 3950 teeth number 14-16; 24-26; 34-36; 44-46) of the individual mouth. At least the posterior parts (4,5) of the first and second U-form half splints (2,3) are hermetically assembled, and the splint (1) is devoid of proximal and distal protuberances. This configuration prevents the mandible from tilting down and the mouth from opening during wearing.

As used herein, the term “proximal” refers to a direction towards the center of the patient body and the term “distal” refers to a direction away from the center of the patient body.

As shown in FIGS. 1 to 6, the splint has substantially a U-form shape which follows the user's internal and external teeth contour.

The plastic material for the monobloc splint (1) of the invention may be any material which is suitable for the purpose. The material must be biocompatible and physiologically safe. It must fulfil the requirements of EU regulation 2017/745 with regard, inter alia, the basic safety and performance requirements. In one embodiment, the plastic material is polyester. In a further embodiment, the plastic material is a thermoforming polyester sheet of the brand Erkodur®, such as the Erkodur® CE No 595120, color glassy.

In an embodiment of the invention, the first and second U-form half splints (2,3) are made of a single plastic material with a single hardness grade. The plastic material has a shore D hardness grade which is preferably higher than 85 and lower than 100. More preferably the shore D hardness grade is about 90.

In one embodiment, the first and second U-form half splints (2,3) are a monobloc single molded component.

In an embodiment of the invention, the first and second U-form half splints (2,3) are hermetically assembled to each other and form a joint line (Z) in the canine, premolar and molar region of the individual (ISO 3950 teeth number 13-17; 23-27; 33-37; 43-47).

In accordance with another embodiment of the invention, the monobloc orthosis splint (1) comprises a front opening (6) between the first and second U form half splints (2,3) in the lateral and central incisor region (ISO 3950 teeth number 11-12; 21-22; 31-32; 41-42). The size of opening (6) is small enough not to enable mouth breathing and its purpose is to allow slight circulation of air, so to avoid a vacuum effect which could cause firm adhesion of the splint onto the teeth. The absolute sizes depend on the specific characteristics of the patient and they may typically vary between 0.05 and 0.5 mm height and between 2 and 5 cm length. The opening (6) can also be used to position the front part of the tongue while the splint (1) is used, and the mouth is closed. The opening (6) also facilitates displacing the monobloc orthosis splint (1) out of the positioning by slight pressure of the tongue against it.

In an embodiment, the monobloc orthosis splint (1) of the invention is suitable exclusively for preventing mouth-breathing in an individual and it is not suitable to correct or compensate structural dental anomalies, such as tooth misalignments or protrusion, bruxism, or to be used as mouth protection in medical or sport applications or for aesthetic purposes such as dental bleaching.

The monobloc orthosis splint (1) of the invention enables to maintain the position of the cross-over point, according to which the incisal edges of the incisors and the canine tips face each other in the static occlusion. But also in the dynamic occlusion, which occurs by the movement of the lower jaw, the splint (1) has to keep the mouth closed. In both static and dynamic occlusions, the tongue is part of this dynamic movement system and is slightly pressed against the palate.

The monobloc orthosis splint (1) of the invention may be produced in the following way: As a first step, the so-called “final bite” has to be determined which represents the natural dentition in the rest position. A specialized dental laboratory determines such “final bite” with an exact fit in the articulator. The point of reference is always the centric relation, which is unique for each individual.

Once the natural dentition in the rest position is determined, the first and second U-form half splints (2,3) are cast from superhard plaster. The first and second U-form half splints (2,3) so obtained are then inserted into the articulator and their height is adjusted in function of the individual natural dentition. This is because the monobloc orthosis splint (1) must guarantee the natural dentition position, both in the static and dynamic rest position of the individual. In an embodiment, the plastic material is Erkodur® CE No 595120 and is 1 mm thick for both the first and second U-form half splints. Only when both half splints are inserted into the articulator and sealed, the monobloc splint is pulled slightly deeper during this working process, in order to match, as closed as possible, the characteristic of the natural dentition of the individual, in one embodiment 1.5 mm.

Depending on the individual dental situation, i.e. natural teeth, implants, crowns, prothesis, partial or full dentures, the first and second U-form half splints (2,3) are sealed together by means of a sealing resin, such as a self-curing acrylic resin (Palapress® CE 0197, Kulzer GmbH), at least in correspondence of the molar and premolar region (ISO 3950 teeth number 14-16; 24-26; 34-36; 44-46) but, alternatively, in correspondence of the canine, premolar and molar region (ISO 3950 teeth number 13-17; 23-27; 33-37; 43-47).

Alternatively, any other sealing technology can be used instead, such as for example direct heat sealing without use of any additional sealing resin.

The so obtained monobloc orthosis splint (1) undergoes a final control check in the articulator. The dental technician has to make sure that the splint (1) allows both the static and the dynamic occlusion of the mouth. The splint must also allow teeth contact without moving the lower jaw in its rest position.

The design of the monobloc orthosis splint (1) of the invention adapts to the anatomy and physiognomy of the individual dentition. Normally, the region corresponding to the incisors of the upper jaw is positioned just in front of that corresponding to the lower jaw. The first and second U-form half splint (2,3) of the splint (1) are slightly offset.

The monobloc orthosis splint (1) of the invention prevents the individual from breathing through open, half open or slightly open mouth. Accordingly, the individual is forced to breathe through the nose and the diaphragm, hence reducing or stopping shortness of breath, gasping breath, shallow breathing, dyspnoea, and breathlessness.

The monobloc orthosis splint (1) of the invention counteracts hypoxemia, hypocapnia and hypoxia. It can be used as a non-medicinal therapeutic and/or as an auxiliary measure for treating and/or reducing symptoms related to inflammatory, obstructive and chronic lung diseases. It can be used for treating and/or reducing Chronic Obstructive Pulmonary Disease (COPD), asthma, including allergic asthma, sleep apnoea, fatigue syndrome, allergies, hypertension, long-lasting COVID-19 symptoms and snoring. In one embodiment, the monobloc orthosis splint (1) can be used for treating and/or reducing Chronic Obstructive Pulmonary Disease (COPD). In another embodiment, the monobloc orthosis splint (1) can be used for treating and/or reducing snoring.

The monobloc orthosis splint (1) of the invention stops physical reflexes to open the mouth, both during sleep and in the resting position when lying, sitting and standing, but also during sport activities, under strain when walking and running and under stress.

The monobloc orthosis splint (1) of the invention helps to reactivate the memory effect of the human organism to breathe like a new born (rebirth breathing). With the help of the monobloc orthosis splint (1) of the invention, the body is immediately able to assume infant breathing.

The monobloc orthosis splint (1) of the invention enables the individual to use, with each breath, the volume of dead space between the nose, the throat, and the bronchi. In an adult, there is an average of 150 to 200 ml of air enriched with carbon dioxide in this anatomical dead space. With each breath, the individual wearing the monobloc orthosis splint (1) of the invention will rebreathe approximately 30% of air from the dead space, before the whole amount of inhaled air will be directed to the alveoli of the lungs. The concentration of the alveolar air remains in balance. The CO₂ content in the whole body remains constant.

Since the monobloc orthosis splint (1) of the invention keeps the mouth closed, the air cannot flow directly to the alveoli. The stress- and panic-inducing hyperventilation is therefore prevented.

The use of the monobloc orthosis splint (1) of the invention leads to the following positive outcome:
Improved oxygen absorption: On average, 10 to 15% percent more oxygen can be absorbed. Nitric oxide generated in the sinuses of the nose is responsible for this.
Improved blood gases: The oxygen supply to the cells is activated. Shortness of breath can be minimized; hyperventilation can be stopped. The body relaxes, comes to more serenity and calmness.
The body moves from an acidic to a neutral pH range through physiological breathing.
Stress levels and panic attacks can be significantly reduced, normalizing high blood pressure, pulse and heartbeat.
The spine of the lower back becomes more stable.
Less tension in the neck-shoulder area.7.
Change of digestion (More nutrients can be absorbed)

### Example

### Scientific Investigation and Evaluation of a Cardiopulmonary Exercise Testing (CPET) Carried Out on an Individual Wearing the Monobloc Orthosis Splint of the Invention

A female patient (54 years old, 1.66 m and 61 kg) diagnosed with a COPD disease (Group Gold III, Group A) underwent a cardiopulmonary exercise testing (CPET). The patient pedaled at 0 W for 1 min and, subsequently, the work rate was increased by 5 W/min until reaching the Max Watt value of 32 W (determined in function of the sex, age, height and weight of the patient, in accordance with the European Respiratory Society Guidelines). The oxygen (O₂), among other parameters, was determined both at rest and at the Max Watt.

During the analysis, the patient wore the monobloc orthosis splint of the invention as described above.

Table 1 shows the O₂ intake at rest (l/min) and at Max Watt (l/min) of a reference (healthy) patient, of a reference COPD GOLD III Group A patient (without wearing the monobloc orthosis splint of the invention) and of the COPD GOLD III Group A patient of the present example (wearing the monobloc orthosis splint of the invention). The values for the Reference Patient have been obtained from the CPET patient database of the Lungenklinik Oberhausen, Germany.

| | O₂ intake at rest | O₂ intake at Max Watt | |
|---|---|---|---|
| Patient Type | (l/min) | (l/min) | |
| Reference Patient | 0.40 | 1.25 | (100%) |
| GOLD III Group A patient | 0.2 | 0.85 | (65%) |
| (without orthosis) | | | |
| GOLD III Group A patient | 0.26 | 1.258 | (101%) |
| (with orthosis) | | | |

The data in FIG. 1 shows that the GOLD III Group A patient wearing the monobloc orthosis splint of the invention has a pulmonary function at rest which is increased by 15% compared to a patient of the same group not wearing the monobloc orthosis splint. When undergoing the effort test as described in the present example, the same patient, who would have a pulmonary function reduced by 35%, performs like the healthy reference patient, or even slightly better.

The monobloc orthosis splint of the invention is a comfort device which can be kept in place even during activities which require effort by forcing nose breathing with the consequent increase of the pulmonary function up to the levels of healthy patients.

1. Monobloc orthosis splint (1) for preventing mouth-breathing in an individual while his temporomandibular joint is in the rest position, comprising a first U-form half splint for the upper jaw (2) and a second U-form half splint for the lower jaw (3);
both the first and second U-form half splints (2,3) being made, at least partially, of plastic material and comprising posterior parts (4,5) intended to be positioned in the molar and premolar region (ISO 3950 teeth number 14-16; 24-26; 34-36; 44-46) of the individual mouth, at least the posterior parts (4,5) of the first and second U-form half splints (2,3) being hermetically assembled, characterized in that the splint (1) is devoid of proximal and distal protuberances.

2. The monobloc orthosis splint (1) according to claim 1, wherein the first and second U-form half splints (2,3) are hermetically assembled in the canine, premolar and molar region (ISO 3950 teeth number 13-17; 23-27; 33-37; 43-47).

3. The monobloc orthosis splint (1) according to any preceding claim, comprising a front opening (6) between the first and second U form half splints (2,3) in the lateral and central incisor region (ISO 3950 teeth number 11-12; 21-22; 31-32; 41-42).

4. The monobloc orthosis splint (1) according to any preceding claim, wherein the first and second U-form half splints (2,3) are made of a single plastic material with a single shore D hardness grade.

5. The monobloc orthosis splint (1) according to any preceding claim, wherein the first and second U form half splints (2,3) are made of a single plastic material having a shore D hardness grade higher than 85 and lower than 100.

6. The monobloc orthosis splint (1) according to any preceding claim, wherein the single plastic material is a polyester.

7. The monobloc orthosis splint (1) according to any preceding claim, wherein the first and second U-form half splints (2,3) are a monobloc single molded component.

8. The monobloc orthosis splint (1) according to any claims 1 to 6, wherein the first and second U-form half splints (2,3) are hermetically assembled to each other along the joint line (Z).

9. The monobloc orthosis splint (1) according to any preceding claim, which is not suitable to correct or compensate structural dental anomalies, such as tooth misalignments or protrusion, bruxism, or to be used as mouth protection in medical or sport applications or for aesthetic purposes such as dental bleaching.

10. The use of the monobloc orthosis splint (1) of any preceding claim, for urging breathing through the nose in an individual or for preventing breathing through the mouth in an individual.

11. The use of the monobloc orthosis splint (1) of any claims 1 to 9 for reducing or stopping shortness of breath, gasping breath, shallow breathing, dyspnoea and breathlessness, or for treating or reducing symptoms associated with Chronic Obstructive Pulmonary Disease (COPD), asthma, including allergic asthma, sleep apnoea, fatigue syndrome, allergies, hypertension, long-lasting COVID-19 symptoms and snoring.

12. The monobloc orthosis splint (1) of any claims 1 to 9 for use in reducing or stopping shortness of breath, gasping breath, shallow breathing, dyspnoea, and breathlessness, or for use in treating or reducing symptoms associated with Chronic Obstructive Pulmonary Disease (COPD), asthma, including allergic asthma, sleep apnoea, fatigue syndrome, allergies, hypertension, long-lasting COVID-19 symptoms and snoring.

13. A method for urging breathing through the nose in an individual or for preventing breathing through the mouth in an individual, comprising the individual using the monobloc orthosis splint of any claims 1 to 9.

14. A method for reducing or stopping in an individual shortness of breath, gasping breath, shallow breathing, dyspnoea, and breathlessness; or for treating or reducing in an individual symptoms associated with Chronic Obstructive Pulmonary Disease (COPD), asthma, including allergic asthma, sleep apnoea, fatigue syndrome, allergies, hypertension, long-lasting COVID-19 symptoms and snoring, comprising the individual using the monobloc orthosis splint of any claims 1 to 9.
